(19) United States
(12) Patent Application Publication
(10) Pub. No.: US 2020/0355691 A1
(43) Pub. Date: Nov. 12, 2020

(54) Direct Colorimetric Detection of Spermine using Gold Nanoparticles

(71) Applicant: New Life Medicine Technology Company Limited, Hong Kong (CN)

(72) Inventors: Ka-Wing WONG, Hong Kong (CN); Tik Hung TSOI, Hong Kong (CN); Chun Wai KAN, Hong Kong (CN)

(21) Appl. No.: 15/929,495

(22) Filed: May 6, 2020

Related U.S. Application Data
(60) Provisional application No. 62/843,604, filed on May 6, 2019.

(51) Int. Cl. G01N 33/574; G01N 21/78; G01N 33/543; G01N 33/553

(52) U.S. Cl. CPC G01N 33/57434; G01N 33/553; G01N 33/54346; G01N 21/78

(57) ABSTRACT

Provided herein are gold nanoparticle based colorimetric methods for the detection of spermine in a sample. The methods can be used to diagnose prostate cancer in a subject based on the detected amount of spermine in a urine sample obtained from the subject.

### CROSS-REFERENCE TO RELATED APPLICATIONS

This application claims the benefit of priority of U.S. Provisional Application No. 62/843,604, filed on May 6, 2019, the contents of which are hereby incorporated by reference in their entirety for all purposes.

### TECHNICAL FIELD

The present disclosure generally relates to gold nanoparticles (AuNPs) and their use in spermine (Spm) detection and cancer diagnosis.

### BACKGROUND

In recent years, a great deal of attention has been focused on the study of a group of biological polyamines, e.g. putrescine, spermidine and Spm. Spm a polycation derived from amino acids, has an important role in governing immune response, neuron regulation and certain pathological events owing to their positively charged nature to easily bind cellular components and therefore regulate the cell physiology. In a recent study, urinary Spm was shown to be useful as a biomarker for the differentiation between prostate cancer and non-malignant benign prostatic hyperplasia (BPH) patients. Other than prostate cancer, Spm is also widely reported as a potential cancer biomarker for a series of cancers, which has increased interest in the development of improved methods for Spm detection.

Conventional methods for quantitative detection of urinary Spm, include chromatographic and electro-migration methods coupled with different detectors, for example, ultra-performance liquid chromatography-tandem mass spectrometry (UPLC-MS/MS), chemiluminescence based high-performance liquid chromatography (HPLC), etc. However, the above techniques require costly instruments, complicated sample preparation and well-trained technical staff for operation.

The application of known probes and methods for detecting urinary Spm are limited by the complex matrix and composition of urine, which poses challenges in sample preparation/clean-up for efficient detection. Recently, AuNPs have been used in a number of applications, including chemical sensing and cellular imaging based on their unique aggregation induced photophysical properties. However, aggregation-based sensing strategies using AuNPs remain challenging due to stability problems, where aggregation can easily occur in biological samples, such as urine, which can lead to false negative results.

Accordingly, there exists a need to develop improved AuNPs based methods for detecting urinary polyamines in complex matrixes, such as urine.

### SUMMARY

Provided herein are methods utilizing thiolate-protected gold AuNPs that exhibit improved stability, high sensitivity, and excellent selectivity for Spm. The practicality of the methods described herein were validated for the detection of Spm in clinical urine samples for cancer screening.

The present disclosure provides a series of biosensors useful for determining Spm concentration in a sample and cancer diagnosis. More particularly, provided herein is citrate-capped AuNPs, which are stabilized and sensitivity-modulated by the presence of a mercapto-bearing agent, such as mercaptoundecanoic acid. Without wishing to be bound by theory, it is believed that the citrate ligands interact with Spm via electrostatic forces causing the AuNPs to be drawn together and aggregate, which induces red-shifting of the surface plasmon resonance (SPR) related absorption to longer wavelengths. Accordingly, the Spm induced aggregation of the AuNPs described herein can be observed by a significant color change of the AuNPs suspension from red to blue/purple due to the presence of Spm real time in a sample and provide a convenient means and straight forward means for detection and quantification.

In a first aspect, provided herein is a method for detecting spermine (Spm) in a sample, the method comprising: providing a sample; contacting the sample with a plurality of gold nanoparticles (MUA-AuNPs) comprising citrate and 11-mercaptoundecanoic acid (MUA) thereby forming a test sample having a test color; and detecting based on the test color the presence of Spm in the sample.

In a first embodiment of the first aspect, provided herein is the method of the first aspect, wherein the sample comprises urine obtained from a subject.

In a second embodiment of the first aspect, provided herein is the method of the first aspect, wherein the step of detecting based on the test color comprises visual inspection of the test color or determining the absorbance of the test sample using a spectrometer.

In a third embodiment of the first aspect, provided herein is the method of second embodiment of the first aspect further comprising the step of comparing the test color with a color chart or color wheel prepared by using the interrelation between color of known concentrations of Spm in standard samples comprising MUA-AuNPs; or comparing the absorbance of the test sample obtained using a spectrometer with one or more calibration curves prepared using the interrelation between absorbance of known concentrations of Spm in standard samples comprising MUA-AuNPs; and determining the concentration of Spm in the sample.

In a fourth embodiment of the first aspect, provided herein is the method of first embodiment of the first aspect, wherein the method further comprises the step of comparing the test color to a color chart or color wheel prepared by using the interrelation between known concentrations of Spm in standard samples comprising MUA-AuNPs; or comparing the absorbance of the test sample using a spectrometer with one or more calibration curves prepared by using the interrelation between known concentrations of Spm in standard samples comprising MUA-AuNPs; determining the concentration of Spm in the sample; and based on the concentration of Spm in the sample determine if the subject has prostate cancer.

In a fifth embodiment of the first aspect, provided herein is the method of first embodiment of the first aspect further comprising the step of: providing a urine sample obtained from the subject; and diluting the urine sample by 50 to 1,000 fold using an aqueous solvent thereby forming the sample.

In a sixth embodiment of the first aspect, provided herein is the method of first embodiment of the first aspect, wherein the aqueous solvent comprises tris(hydroxymethyl)aminomethane (Tris)-HCl and NaCl.

In a seventh embodiment of the first aspect, provided herein is the method of first embodiment of the first aspect, wherein the plurality of MUA-AuNPs are prepared according to a method comprising: contacting HAuCl₄ with trisodium citrate thereby forming a plurality of citrate capped gold nanoparticles (citrate-AuNPs); and contacting the plurality of citrate-AuNPs with 11-mercaptoundecanoic acid thereby forming the plurality of MUA-AuNPs.

In an eighth embodiment of the first aspect, provided herein is the method of seventh embodiment of the first aspect, wherein HAuCl₄ and trisodium citrate are contacted in a molar ratio between 1:3 to 1:15, respectively.

In a ninth embodiment of the first aspect, provided herein is the method of seventh embodiment of the first aspect, wherein the citrate-AuNPs and MUA are contacted in a molar ratio of 1:3 to 1:15, respectively.

In a tenth embodiment of the first aspect, provided herein is the method of first embodiment of the first aspect, wherein the after the step of contacting the sample with the plurality of MUA-AuNPs the test sample is incubated for at least 15 minutes before the step of detecting based on the test color the presence of Spm in the sample.

In an eleventh embodiment of the first aspect, provided herein is the method of first embodiment of the first aspect, wherein after the step of contacting the sample with the plurality of MUA-AuNPs the test sample is incubated for 15 and 30 minutes before the step of detecting based on the test color the presence of Spm in the sample.

In a twelfth embodiment of the first aspect, provided herein is the method of the first aspect, wherein the method comprises: providing a sample comprising urine at a concentration of 0.1-2% (v/v), wherein the urine was obtained from a subject; contacting the sample with a plurality of MUA-AuNPs comprising citrate and MUA thereby forming a test sample having a test color; and detecting based on the test color the presence of Spm in the sample, wherein after the step of contacting the sample with the plurality of MUA-AuNPs, the test sample is incubated for 15 and 30 minutes before the step of detecting based on the test color the presence of Spm in the sample.

In a thirteenth embodiment of the first aspect, provided herein is the method of twelfth embodiment of the first aspect, wherein the plurality of MUA-AuNPs have an average particle size of 1-20 nm.

In a fourteenth embodiment of the first aspect, provided herein is the method of twelfth embodiment of the first aspect, wherein the sample comprises Tris-HCl and NaCl.

In a fifteenth embodiment of the first aspect, provided herein is the method of twelfth embodiment of the first aspect, wherein the plurality of MUA-AuNPs are prepared according to a method comprising: contacting HAuCl₄ with trisodium citrate in a molar ratio of 1:3 to 1:15, respectively thereby forming a plurality of citrate-AuNPs; and contacting the plurality of citrate-AuNPs with MUA in a molar ratio of 1:3 to 1:15, respectively, thereby forming the plurality of MUA-AuNPs.

In a second aspect, provided herein is a method for diagnosing prostate cancer in a subject, the method comprising: providing a sample comprising urine at a concentration of 0.1-2% (v/v), wherein the urine was obtained from the subject; contacting the sample with a plurality of MUA-AuNPs comprising citrate and MUA thereby forming a test sample having a test color; and determining based on the test color whether the subject has prostate cancer, wherein after the step of contacting the sample with the plurality of MUA-AuNPs the test sample is incubated for 15 and 30 minutes before the step of determining based on the test color whether the subject has prostate cancer.

In a first embodiment of the second aspect, provided herein is the method of the second aspect, wherein the step of determining based on the test color whether the subject has prostate cancer comprises comparing the test color to a color chart or color wheel prepared by using the interrelation between known concentrations of Spm in standard samples comprising MUA-AuNPs; or comparing the absorbance of the test sample using a spectrometer with one or more calibration curves prepared by using the interrelation between known concentrations of Spm in standard samples comprising MUA-AuNPs; determining the concentration of Spm in the sample; and based on the concentration of Spm in the sample determine if the subject has prostate cancer.

In a second embodiment of the second aspect, provided herein is the method of the second aspect, wherein the plurality of MUA-AuNPs are prepared according to a method comprising: contacting HAuCl₄ with trisodium citrate in a molar ratio of 1:3 to 1:15, respectively thereby forming a plurality of citrate-AuNPs; and contacting the plurality of citrate-AuNPs with MUA in a molar ratio of 1:3 to 1:15, respectively thereby forming the plurality of MUA-AuNPs.

In a third embodiment of the second aspect, provided herein is the method of the second aspect, wherein the MUA-AuNPs have an average particle size of 10-15 nm.

### BRIEF DESCRIPTION OF THE FIGURES

The above and other objects and features of the present disclosure will become apparent from the following description of the disclosure, when taken in conjunction with the accompanying drawings.

FIG. 1 shows Table 1 shows Spm detection (μM) results from clinical urine samples by the MUA-AuNPs of FIG. 4A and LC-MS/MS (n=100).

FIG. 2 shows a representative illustration of colorimetric Spm detection using the MUA-AuNPs described herein.

FIG. 3 shows a representative illustration of the preparation of MUA-AuNPs and aggregation based on electrostatic interactions between MUA-AuNPs and Spm.

FIG. 4A shows a transmission electron microscope (TEM) image of spherical shaped MUA-AuNPs that are red in color in the absence of Spm.

FIG. 4B shows a TEM image of the MUA-AuNPs of FIG. 4A in the presence of Spm (250 nM) and are purple in color.

FIG. 5 shows the typical response of the MUA-AuNPs of FIG. 4A to urine samples contain varying concentrations of Spm under different incubation times.

FIG. 6 shows a chromatic illustration of different concentrations of Spm (0-500 nM) in water (2.5 uL) added to 235 uL MUA-AuNP solutions. (5 mM Tris 50 mM NaCl pH 7.2).

FIG. 7 shows a typical UV-visible absorption spectrum (hereinafter, ‘spectrum’) of spherical MUA-AuNPs having a SPR absorption around 525 nm being red in color, which undergoes a spectral shift upon addition of Spm (0-200 nM).

FIG. 8A shows a plot of the changes in absorbance ration upon interaction of MUA-AuNPs with Spm (0-200 nM).

FIG. 8B shows a plot of Abs₆₁₀/Abs₅₂₇ vs Spm concentration.

FIG. 9 shows a typical spectrum of MUA-AuNPs across different polyamines (250 nM). MUA-AuNPs only show a SPR absorption shift in the presence of Spm.

FIG. 10 shows a plot of normalized Abs610/Abs527 of the MUA-AuNPs described herein vs SPM concentration (60-160 nM).

FIG. 11 shows pictures of the optimization of sample dilution studies.

FIG. 12 shows a UV-visible absorption spectrum of citrate-AuNPs prepared from different concentrations of trisodium citrate.

FIG. 13 shows a UV-visible absorption spectrum of MUA-AuNPs prepared using different concentrations of trisodium citrate.

FIG. 14 shows pictures of MUA-AuNPa prepared using different concentrations of MUA in the MUA—citrate ligand exchange reaction.

### DETAILED DESCRIPTION

The methods for detecting Spm described herein relies on the unique optical properties of AuNPs (e.g., having dimensions ranging from 1-100 nm) due to their size and morphology. Small spherical AuNPs (˜30 nm) can absorb light in the blue-green region of the visible spectrum (450-550 nm) while red light (˜700 nm) is reflected, yielding a rich red color. As the AuNPs size increases or aggregation of the AuNPs occurs, the larger particles can exhibit increased scattering and broadened absorption peaks and red-shifting of the SPR related absorption to longer wavelengths. This phenomenon can be observed by a change in color of AuNPs suspensions resulting from aggregation.

The present disclosure provides highly sensitive AuNPs based sensors for rapid and direct detection of Spm in a sample, which advantageously is colorimetric allowing detection to be determined visually or in the alternative using a low cost spectrometer.

The methods provided herein are capable of selectively detecting Spm in a sample (such as urine) with nanomolar sensitivity in as little as 15 to 30 minutes, which is improvement over existing methods. In addition, the assay can be performed easily without training, which could be operated by any unskilled individuals. The sensor can be readily prepared from commercially available starting materials agents in a two-step process. The MUA-AuNPs are highly stable in the buffer system described herein and can be stored conveniently.

The methods described herein utilize MUA-AuNPs comprising citrate or a conjugate acid thereof; and MUA or a conjugate base thereof.

Citric acid and MUA contain 4 and 2 acidic protons, respectively, and can thus exist in multiple protonated states. The MUA-AuNPs described herein can comprise citrate and MUA in any protonation state and mixtures thereof. The protonation state can depend on the pH of the composition comprising the MUA-AuNPs.

The MUA-AuNPs described herein can be prepared using any number of well-known methods in the art. All such methods are contemplated by the present disclosure. In certain embodiments, the MUA-AuNPs are prepared in a two-step process comprising reduction of a gold salt by citric acid or a salt thereof thereby forming a citrate-AuNPs followed by ligand exchange with a mercaptan containing compound thereby yielding the MUA-AuNPs.

The citrate-AuNPs can be prepared by reducing a gold salt with a citrate reducing agent selected from citric acid or a salt thereof. In certain embodiments, the gold salt is Au(I), Au(II), Au(III), or a mixture thereof. The gold salt can comprise anions selected from the group consisting of chloride, bromide, iodide, nitrate, hydroxide, PF₆, BF₄, mesylate, triflate, cyanide, acetate, and the like, and combinations thereof. In certain embodiments, gold salts suitable for preparing the citrate-AuNPs described herein can be represented by the formula: MAuX₄, wherein M is H, Li, Na, and NH₄; and X for each instance is independently Cl, Br, or I. Exemplary gold salts suitable for preparing the citrate-AuNPs described herein include, but are not limited to, HAuCl₄ and HAuBr₄.

In certain embodiments, the citrate reducing agent can be represented by the formula: (CO₂M)CH₂C(OH)(CO₂M)CH₂(CO₂M), wherein M for each instance is independently selected from H, Li, Na, and NH₄. In certain embodiments, the citrate reducing agent is trisodium citrate, trilithium citrate magnesium citrate, calcium citrate or a mixture thereof.

In the examples below, the citrate-AuNPs used in the methods described herein are directly produced by a wet chemical method comprising reduction of chloroauric acid (HAuCl₄) with trisodium citrate in an aqueous solvent (e.g., water) thereby yielding monodisperse citrate-AuNPs.

The citrate reducing agent is typically used in excess amount in the reduction of the gold salt. Depending on the oxidation state of the gold salt, the citrate reducing agent can be used in a molar ratio between 1 to 20 relative to the gold salt.

In certain embodiments, the molar ratio of the citrate reducing agent to the gold salt is between 2:1 to 20:1; 2:1 to 19:1; 2:1 to 18:1; 2:1 to 17:1; 2:1 to 16:1; 2:1 to 15:1; 3:1 to 15:1; or 3:1 to 13:1. In certain embodiments, between 155-1,240 mol % or 310-1,240 mol % of trisodium citrate is used to reduce HAuCl₄.

The reduction of the gold salt can be conducted in an aqueous solvent. The reduction can be conducted at a temperature between 23-100° C. In certain embodiments, the reduction is conducted at a temperature between 60-100° C., 40-100° C., 80-100° C., 90-100° C., or 100° C.

The average size of the citrate-AuNPs prepared according to the methods described herein can range from 1 to 20 nm. In certain embodiments, the average size of the citrate-AuNPs is 5 to 20, 7 to 20, 10 to 20, 10 to 17, or 10 to 15 nm. In certain embodiments, the average size of the citrate-AuNPs is about 13 nm. In certain embodiments, a suspension of the citrate-AuNPs can have the average size of about 13 nm with the possibility that the suspension including citrate-AuNPs that range from 10 to 50 nm.

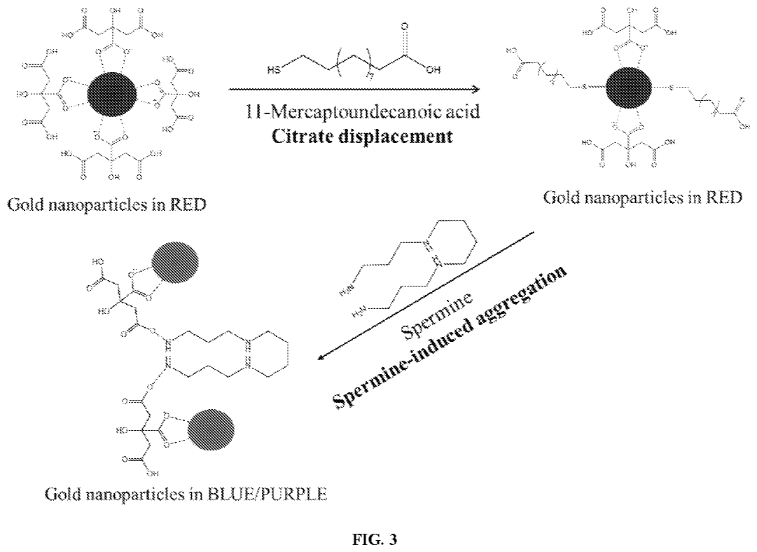

MUA-AuNPs can be prepared from the citrate-AuNPs by a ligand exchange reaction in which one or more citrate ligands bound to the surface of the citrate-AuNPs is substituted with one or more MUA ligands thereby forming a plurality of MUA-AuNPs comprising citrate and MUA ligands. FIG. 3 depicts a representative reaction sequence showing the preparation of the MUA-AuNPs and the displacement of surface bound citrate ligands with MUA thereby forming the plurality of MUA-AuNPs.

The citrate-AuNPs ligand exchange reaction can be conducted in an aqueous solvent (e.g., water).

MUA can be used in a molar ratio between 1 to 20 relative to the citrate-AuNPs (stoichiometry based on the assumption that 100% of the gold salt is converted to citrate-AuNPs and relative to gold). In certain embodiments, the molar ratio of the MUA to citrate-AuNP is between 2:1 to 20:1; 2:1 to 19:1; 2:1 to 18:1; 2:1 to 17:1; 2:1 to 16:1; 2:1 to 15:1; 3:1 to 15:1; or 3:1 to 13:1. In certain embodiments, between 155-1,240 mol % or 310-1,240 mol % of MUA is used in the ligand exchange reaction with the citrate-AuNPs.

In certain embodiments, the average size of the MUA-AuNPs ranges from 1 to 20 nm. In certain embodiments, the average size of the MUA-AuNPs is 5 to 20, 7 to 20, 10 to 20, 10 to 17, or 10 to 15 nm. In certain embodiments, the average size of the citrate-AuNPs is about 13 nm.

The method for detecting Spm in a sample can comprise: providing a sample; contacting the sample with a plurality of MUA-AuNPs comprising citrate and MUA thereby forming a test sample having a test color; and detecting based on the test color the presence of Spm in the sample.

The sample may contain the target Spm. Such a sample may include a water sample, a food sample or a biological sample obtained from plants or animals, or from body fluid of a subject (such as urine), including a mammal, such as a human. The sample may be a “clinical sample,” which is a sample derived from a patient such as urine. In certain embodiments, the sample comprises urine obtained from a human subject.

The sample can comprise urine obtained from the subject, which has been diluted between by 5 to 1,000 fold using an aqueous solvent (i.e., by adding 1 part urine to 4 parts aqueous solvent to 1 part urine to 999 parts aqueous solvent). In certain embodiments, the urine can be diluted between 5 to 1,000 fold; 5 to 500 fold; 10 to 500 fold; 10 to 400 fold; 10 to 300 fold; 10 to 200 fold; 20 to 200 fold; 30 to 200 fold; 40 to 200 fold; 50 to 200 fold; 50 to 150 fold; 50 to 125 fold; 50 to 100 fold; 70 to 120 fold; 80 to 120; 90 to 120 fold; or 90 to 110 fold with the aqueous solvent. In certain embodiments, the sample comprises between 0.1-10%; 0.1-9%; 0.1-8%; 0.1-7%; 0.1-6%; 0.1-5%; 1-5%; 1-4%; 1-3%; 1-2%; 1-1.5%; 1-1.4%; 1-1.3%; 1-1.1%; 0.2-1.1%; 0.3-1.1%; 0.4-1.1%; 0.5-1.1%; 0.6-1.1%; 0.7-1.1%; 0.8-1.1%; or 0.9-1.1% urine (v/v).

The aqueous solvent used to dilute the urine sample can be buffered. Any buffer system conventionally used for buffering clinical samples can be used to buffer the urine sample. In certain embodiments, the sample comprises a Tris-HCl or (4-(2-hydroxyethyl)-1-piperazineethanesulfonic acid) (HEPES) buffer. In certain embodiments, the sample has a pH between 7.0-7.5; 7.0-7.4; 7.0-7.3; or 7.1-7.3.

The aqueous solvent may also comprise NaCl at a concentration between 1-100 mM. In certain embodiments, the concentration of NaCl in the sample is 10-100, 10-90, 10-80, 20-80, 30-80, 30-70, or 40-60 mM.

The sample can further comprise a preservative, such as NaN₃, for preventing antimicrobial growth. The concentration of the NaN₃ in the sample can range between 0.01 to 0.1% (w/w). In certain embodiments, the concentration of the NaN₃ in the sample ranges between 0.01 to 0.1%; 0.01 to 0.09%; 0.01 to 0.08%; 0.02 to 0.08%; 0.03 to 0.08%; 0.03 to 0.07%; 0.03 to 0.06%; or 0.04 to 0.06% (w/w).

As discussed herein, the MUA-AuNPs can comprise citrate and/or MUA in different protonation states (e.g., conjugate base of MUA and conjugate acids of citrate). All such forms are contemplated in the methods described herein. In instances in which the MUA-AuNPs comprises conjugate acids of citrate, one or two of the carboxylic acids presents in the citrate can exist in protonated form. In instances in which the MUA-AuNPs comprises a conjugate base of MUA, the conjugate may exist as a salt of Na, Li, Mg, Ca, NH₄, or the like.

Detecting the presence of Spm in the sample can be conducted by visual inspection and/or using a spectrometer. Any conventional spectrometer capable of measure absorbance of the test sample, which can fall between about 450 to 700 nm. In certain embodiments, the spectrometer is a visible light spectrometer or an ultraviolet-visible light spectrometer that is capable of measuring absorbance of the test sample between 450 to 700; 500 to 650 nm; 550 to 650 nm; 600 to 650 nm; or 600 to 630 nm.

Due to the marked color difference of the MUA-AuNPs aggregates formed in the presence of Spm, detection of Spm in a sample can also be conducted by simple visual observation of the sample. Depending on the color of the test sample, it can be determined whether Spm is present.

Detecting the presence of Spm in the sample can also comprise determining the concentration of Spm in the sample. In such instances, the method can further comprise comparing the test color with a color chart or color wheel prepared by using the interrelation between color of known concentrations of Spm in standard samples comprising MUA-AuNPs; or comparing the absorbance of the test sample obtained using a spectrometer with one or more calibration curves prepared using the interrelation between absorbance of known concentrations of Spm in standard samples comprising MUA-AuNPs; and determining the concentration of Spm in the sample.

The interrelation between the color of the test sample and MUA-AuNPs in standard samples having known concentrations of Spm can be determined by preparing a series of standard samples, preferably comprising a similar analyte matrix, containing the MUA-AuNPs and known concentrations of Spm (e.g., between 50 to 300 nM Spm) and determining the color of each standard sample having a different Spm concentration. The concentration of the test sample can then be determined by simply comparing the color of the test sample with the colors of each of the standard samples having different concentrations of Spm and determining the concentration of Spm in the standard sample based on which standard sample Spm concentration has the closest color. A color chart, color wheel, or the like indicating the interrelation between test/standard sample color and Spm concentration can be prepared beforehand to simplify this process.

The interrelation between the color of the test sample and MUA-AuNPs in standard samples having known concentrations of Spm can be determined by preparing a series of standard samples, preferably comprising a similar analyte matrix, containing the MUA-AuNPs and known concentrations of Spm (e.g., between 50 to 300 nM Spm) and determining the absorbance of each standard sample having a different Spm concentration using a spectrometer. One or more calibration curves can be prepared using the interrelation between absorbance of known concentrations of Spm in standard samples comprising MUA-AuNPs. The concentration of Spm in the test sample can then be determined by comparing the absorbance of the test sample with the calibration curve.

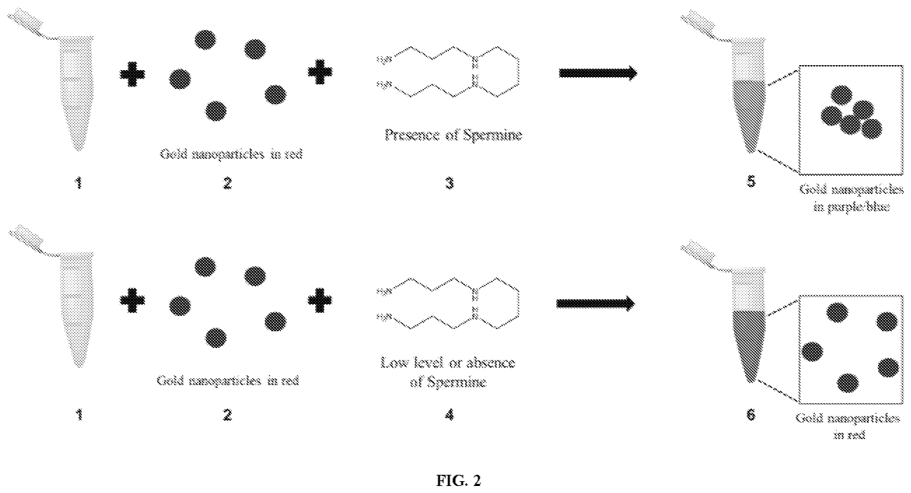

Referring to FIG. 2 the method of detecting Spm comprises: (a) adding a MUA-AuNPs suspension 2 to a buffer 1; and (b) adding a sample suspected of containing the target Spm 3 or 4 to buffer 1, wherein a color change (red to purple) is brought by the sample containing Spm 5. Similarly, wherein no color change is observed (remains red) the sample does not contain or contain only low level of the target Spm 6.

In certain embodiments, the method of detecting Spm comprising the steps of: contacting the MUA-AuNPs described herein with a sample suspected of containing Spm thereby forming a test sample and measuring the absorption of the test sample. In certain embodiments, the step of measuring the absorption is accomplished visually, by comparison with a color wheel or color chart, or by measurement using a spectrometer. In certain embodiments, the step of measuring the absorption of the test sample comprising detecting absorption intensity of the test sample in the range of 450 to 700 nm. In certain embodiments, the step of measuring the absorption comprising detecting absorption intensity of the sample in the range of 500 to 650 nm; 550 to 650 nm; 600 to 650 nm; or 600 to 630 nm.

In certain embodiments, the step of contacting the sample with the plurality of MUA-AuNPs comprises contacting an aqueous solution comprising the plurality of MUA-AuNPs and the sample. Depending on the concentration of Spm in the sample, the concentration of the MUA-AuNPs in the aqueous solvent can vary. The determination of the necessary concentration of MUA-AuNPs in the aqueous solvent can be determined based on the teachings described herein and general knowledge known to a person of ordinary skill in the art. The concentration of the plurality of MUA-AuNPs in the aqueous solvent can be calculated according to the analytical approximation for spherical gold nanoparticles developed by Haiss (2007) and assuming that all citrate-AuNPs are converted to MUA-AuNPs. The concentration of the plurality of MUA-AuNPs in the aqueous solvent can range from 0.742 to 56.6 nmol/L. In certain embodiments, the concentration of the plurality of MUA-AuNPs in the aqueous solvent can range from 0.742 to 1.42 nmol/L (0.25×MUA), 1.484 to 2.84 nmol/L (0.5×MUA), 2.97 to 5.66 nmol/L (1×MUA), 5.94 to 11.32 nmol/L (2×MUA), 11.88 to 22.64 nmol/L (4×MUA), or 29.7 to 56.6 nmol/L (10×MUA). In certain embodiments, the concentration of the plurality of MUA-AuNPs in the aqueous solvent can range from 29.7 to 56.6 nmol/L (10×MUA).

In certain embodiments, the step of contacting the sample with the plurality of MUA-AuNPs comprises adding the plurality of MUA-AuNPs to the sample or adding the sample to the aqueous solution comprising the plurality of MUA-AuNPs. In certain embodiments, an aqueous solvent comprising the plurality of MUA-AuNPs is added to the sample.

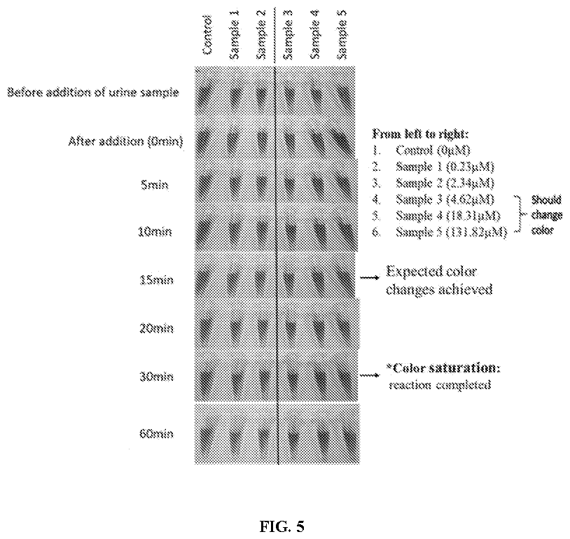

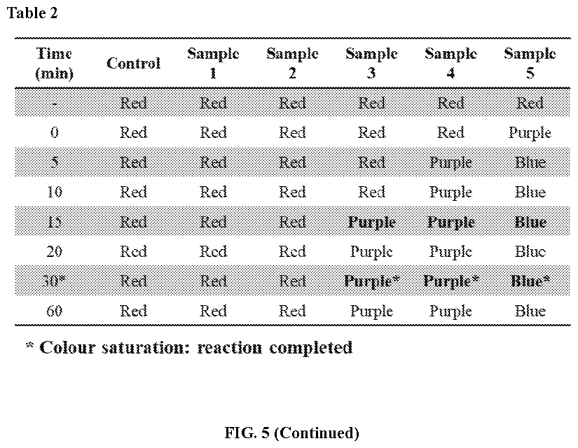

In certain embodiments, the incubation time after contacting the MUA-AuNPs and the sample suspected of containing Spm is 30 minutes or less, at which point the test color of the test sample is examined. In certain embodiments, the incubation time after contacting the MUA-AuNPs with the sample suspected of containing Spm until the test color of the test sample is examined is at least 15 minutes; or 15-30, 15-25, 15-20, 20-30, or 25-30 minutes. As shown in FIG. 5, the methods provided herein can advantageously provide testing results in as little as 5-15 minutes as demonstrated by the time entries at 5, 10, and 15 minutes for Samples 3, 4, and 5 in which only the intensity of the color of the test sample continues to chance but not the absorbance wavelength of the test sample.

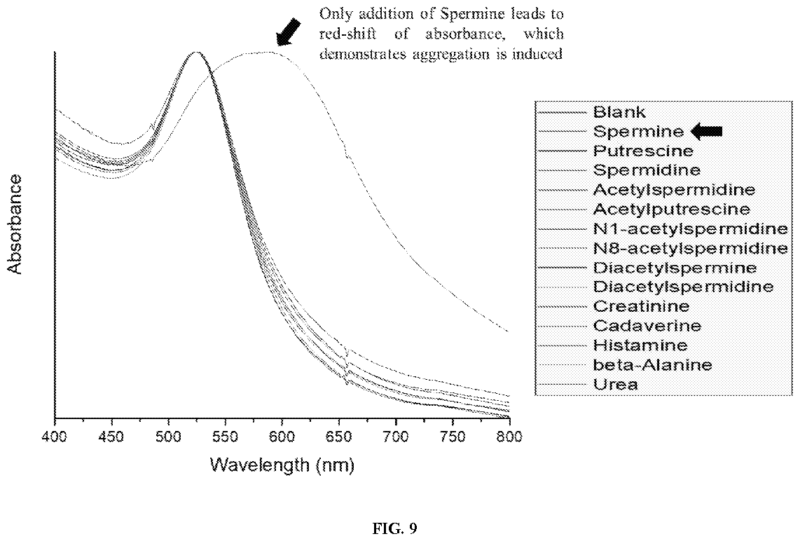

The MUA-AuNPs exhibit a highly selective red-shift in the presence of Spm, which is not observed with other biologic amines, as shown in FIG. 9. Such high selectivity can improve the sensitivity of the method for detecting Spm in complex analyte matrixes, such as urine.

The methods for detecting and quantifying Spm in a test sample comprising urine obtained from a subject can be used to diagnose whether the subject suffers from prostate cancer. In such instances, the methods described herein can further comprise the step of comparing the test color to a color chart or color wheel prepared by using the interrelation between known concentrations of Spm in standard samples; or comparing the absorbance of the test sample using a spectrometer with one or more calibration curves prepared by using the interrelation between known concentrations of Spm in standard samples; determining the concentration of Spm in the sample; and based on the concentration of Spm in the sample determine if the subject has prostate cancer.

In certain embodiments, the method for diagnosing prostate cancer in a subject comprises: providing a sample comprising urine at a concentration of 1-2% (v/v), wherein the urine was obtained from the subject; contacting the sample with a plurality of MUA-AuNPs comprising citrate and MUA or a conjugate base thereof for 15 to 30 minutes thereby forming a test sample having a test color; and determining based on the test color whether the subject has prostate cancer.

In certain embodiments, the step of determining based on the test color whether the subject has prostate cancer comprises comparing the test color to a color chart prepared by using the interrelation between known concentrations of Spm in standard samples comprising MUA-AuNPs; or comparing the absorbance of the test sample using a spectrometer with one or more calibration curves prepared by using the interrelation between known concentrations of Spm in standard samples; determining the concentration of Spm in the sample comprising MUA-AuNPs; and based on the concentration of Spm in the sample determine if the subject has prostate cancer.

Also provided herein is a device for conducting the method for detecting Spm described herein, the device comprising a compartment suitable for containing the incubation solution; and piercing the seconding compartment containing a mixture comprising the MUA-AuNPs such that the sensor mixture comprising the MUA-AuNPs is able to fall into the compartment containing the incubation buffer; a third compartment is used for transferring a sample suspected of containing a target Spm or to the first compartment containing the MUA-AuNPs and incubation buffer, which is capable of demonstrating a visual color change in the presence of the target Spm.

The first compartment of the detection kit could be any alternatives able for carrying fluid as simple as a glass vial or an Eppendorf tube. The second compartment could be any devices that are able to deliver fluid to the system such as a syringe, a pipette or any other suitable means known in the art. Similarly, the third compartment could be any suitable delivery tubes as shown in this embodiment. In general, the detection kit containing all three compartments could be assembled or designed as one single unit, such as a cup or any alternatives.

In certain embodiments, the kit further comprises a color chart or color wheel that can be used to correlate the color of the sample with at least one of a concentration of Spm in the sample or the likelihood that patient is has cancer.

In certain embodiments, the kit further comprises instructions for performing the method of detecting Spm in a sample described herein.

Reagents and Apparatus

Spm, chloroauric acid (HAuCl₄), trisodium citrate, MUA, hydrochloric acid (HCl), nitric acid (HNO₃), tris(hydroxyethyl)aminomethane (TRIS), sodium chloride (NaCl), sodium azide (NaN₃), and sodium hydroxide (NaOH) were obtained from Sigma-Aldrich (Hong Kong, China). Ethanol (EtOH) was obtained from ACROS (USA). All chemicals and reagents were of analytical grade and were used without further purification. Water was purified in a MilliQ Direct Water Purification System (Millipore, USA). The UV-Vis absorption spectra were recorded using a Cary 8453 UV-Vis Spectrometer (Agilent, Hong Kong, China). Dynamic Light Scattering (DLS) was measured by a Zetasizer Nano-ZS90 System (Malvern Instruments, Shanghai, China).

All glassware was cleaned in a bath of freshly prepared 3:1 (v/V) HNO₃-HCl and then rinsed thoroughly with Milli-Q water. 13 nm citrate-AuNPs were firstly prepared by sodium citrate-mediated reduction of HAuCl₄, which was described previously with slight modification. Briefly, HAuCl₄ (4.25 mg, 12.5 μmol) was dissolved in 25.0 mL Milli-Q water (0.50 mM) to form a pale yellow aqueous solution. The solution was heated to reflux under stirring and was then reacted with sodium citrate dihydrate (0.02 g, 68 μmol) in 1.0 mL water (2% w/w) to form a dark purple solution. The resulting solution was stirred for another 20 min at 100° C. until the solution color changed to wine-red indicating the complete formation of citrate-AuNPs. By controlling the amount of citrate in the synthesis, the size of nanoparticles achieved can be controlled in a range of 13 to 50 nm

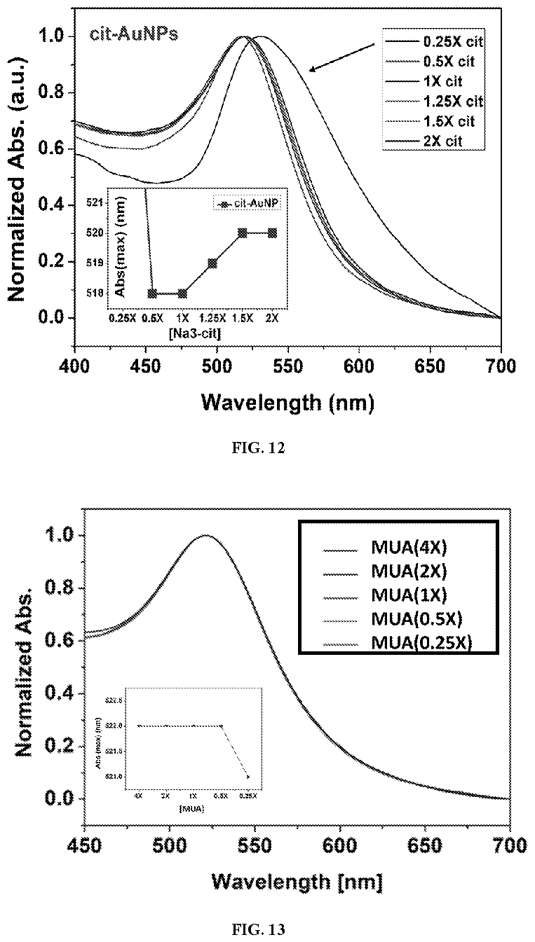

Based on the synthesis of citrate-AuNPs as illustrated above, a further study on the effect of different equivalents of trisodium citrate on the photophysical properties of the prepared citrate-AuNP was carried out. Various equivalents of trisodium citrate (0.25×, 0.5×, 1×, 1.25×, 1.5×, and 2× corresponding to 17, 34, 68, 85, 102 and 136 mol, respectively) were reacted with HAuCl₄ (4.25 mg, 12.5 μmol) in 25.0 mL Milli-Q water (0.50 mM) to prepare citrate-AuNP. The as-prepared citrate-AuNPs were diluted and examined using UV-Vis absorption measurements. As seen in FIG. 12, when 0.25× (17 μmol) citrate was used in the reduction, a red shift in the absorbance of the citrate-AuNP product was observed (Abs max at 532 nm) that indicated comparatively enlarged citrate-AuNPs or the occurrence of aggregation. In contrast, citrate concentrations greater than 0.25× only resulted in a slight change in absorbance indicating little or no aggregation and/or relatively smaller citrate-AuNPs.

Upon completion of the preparation of citrate-AuNPs, the dark wine-red solution was allowed to cool down to room temperature and was transferred to a clean 50 mL Erlenmeyer flask. The volume of the mixture was adjusted to 25.0 mL by addition of water.

In certain embodiments, the pH of the citrate-AuNP suspension is adjusted (e.g., using by NaOH) to 11, followed by dropwise addition of the 11-mercaptoundecanoic acid in 200 μL ethanol to generate MUA-AuNP by the ligand exchange between citrate and thiol-containing agent. The mixture was then stirred overnight, and the residue was collected by centrifugation and redispersion using the minimum amount of the supernatant to yield a concentrated (10X) MUA-AuNP suspension.

The as-obtained citrate-AuNPs obtained from different concentrations of trisodium citrate (except 0.25×) were further reacted with same amount of MUA (i.e., 0.5×, 1×, 1.25×, 1.5×, and 2× corresponding to 34, 68, 85, 102 and 136 mol, respectively). The obtained MUA-AuNPs were diluted and followed by UV-Vis absorption measurements. After MUA modification, the average absorbance shift was around 5 nm and the absorbance shift varied within 1 nm for the same batch of citrate-AuNPs, highlighting the importance of obtaining desired citrate-AuNP (FIG. 13).

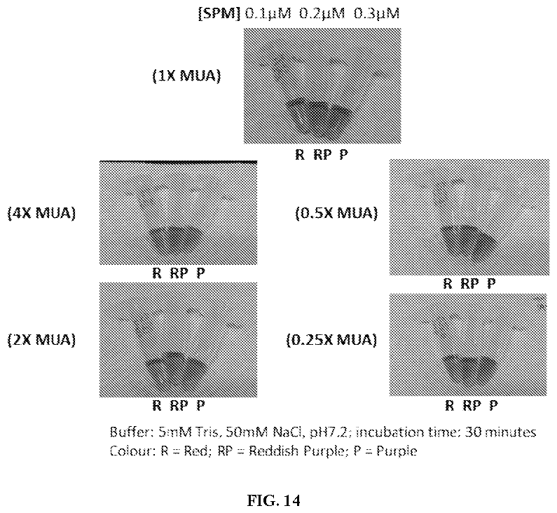

Various concentrations of MUA (0.25×, 0.5×, 1×, 2×, 4× which correspond to 17, 34, 68, 85, 136, 272 mol, respectively) were used in the preparation of MUA-AuNP. The MUA was added directly to the as prepared citrate reduction reaction product mixture. As seen in FIG. 14, doubling or halving the amount of MUA had no impact on the absorbance of the product MUA-AuNPs (in (5 mM Tris, 50 mM NaCl) indicating no differences in aggregation and/or particle size.

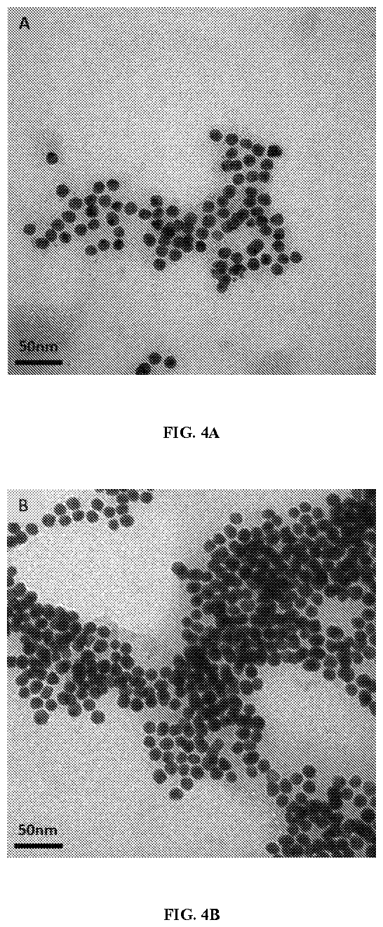

Drops of colloidal MUA-AuNP suspension were placed onto carbon coated copper grids and left to dry at room temperature. The copper grids were than analyzed on a FEI Tecnai G2 20 S-Twin transmission electron microscope with an accelerating voltage of 200 kV (ThermoFisher, Oregon, USA). The performed TEM experiments demonstrate the Spm-induced aggregation profiles of the MUA-AuNP (FIGS. 4a and 4b).

To establish the robustness of the assay, various incubation times after the addition of sample to the MUA-AuNP were tested. This is to ensure that the truly positive samples exhibit colorimetric change at the optimum incubation time and therefore do not result in false negatives. Visual assay is performed under different incubation time (FIG. 5). The assays are pictured at 7 time points (0, 5, 10, 15, 20, 30 and 60 minutes). It was found that the MUA-AuNP aggregated within 15 minutes and saturated in 30 minutes. This demonstrates that the assay should be performed and recorded in 30 minutes for the best accuracy.

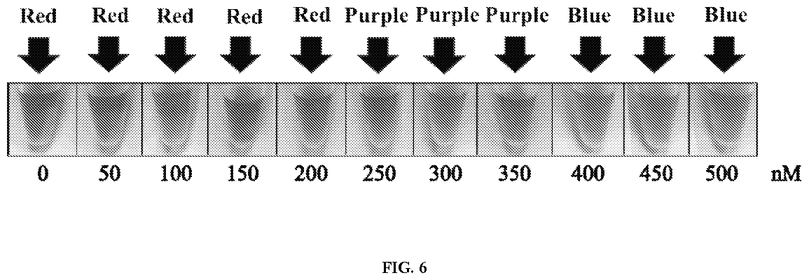

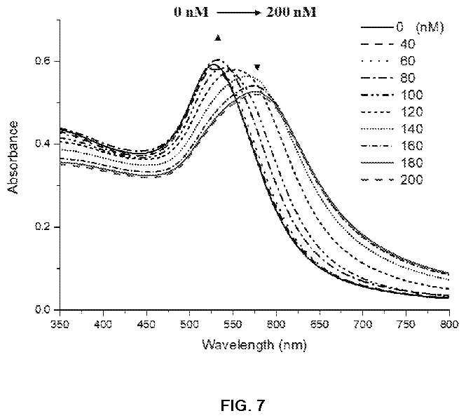

The final sample having a volume of 250 μL containing a buffer comprising 10 mM Tris-HCl, 50 mM NaCl, pH 7.2, and 12.5 μL of 10×MUA-AuNPs. MUA-AuNPs (10×, 12.5 μL) was diluted with 235 μL of 10 mM Tris-HCl (50 mM NaCl, pH 7.2) buffer to yield a 0.5×MUA-AuNPs suspension. Standard solution (Spm) or clinical urine samples (2.5 μL) were added and diluted 100-fold by 0.5×MUA-AuNPs suspensions. The solution was allowed to stand for 30 min incubation followed by an observable color change and UV-Vis absorption measurements (FIG. 6 and FIG. 7).

The MUA-AuNPs of the present disclosure are useful for detecting the concentration of Spm in a sample, such as in a urine sample. In certain embodiments, the method for colorimetric detection of the level of Spm in a sample of urine calls for the following components: (a) a sample of urine; (b) MUA-stabilized citrate-AuNPs (MUA-AuNPs) for detecting visual color change indicates the level of Spm in the sample of urine; and (c) a buffer for the dilution of NUA-AuNPs and the sample of urine.

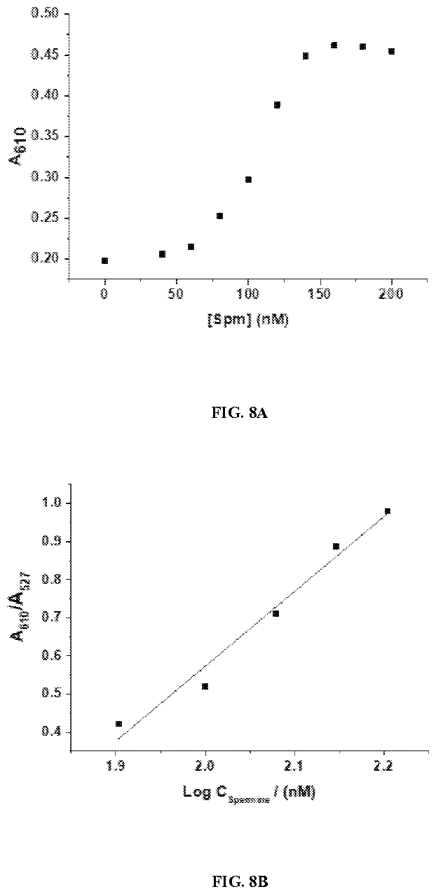

The Spm level in urine may be measured and detected by the visual color change as shown in FIG. 2. Urinary Spm level can be used for distinguishing prostate cancer patient and benign prostatic hypoplasia patient. To obtain the best sensitivity and specificity, the detection threshold is determined to be 3.5 μM. The detection limit of Spm of the present invention can be varied by adjusting the ionic concentration of buffer system 1. The amount of Spm in a sample of urine can be determined by the visual change of color. First the concentrated sensor must be introduced into the buffer system. Second the sample is added to the diluted sensor solution and the amount of Spm in the sample can be quantified for example by using a calibration curved established by titration (FIG. 8).

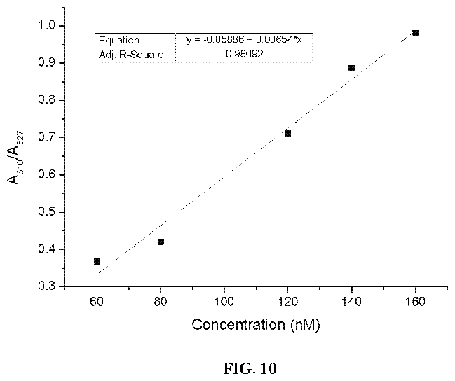

Advantageously, the compositions and methods described herein are able to exhibit a linear relationship between 60-160 nM of Spm. A Spm calibration curve fitted by the least square linear regression. The equation was y=−0.05886+0.00654×, with limit of detection (LOD) calculated to be 60.54 nM by the equation of LOD=3.3*(standard deviation of intercept/slope) (FIG. 10).

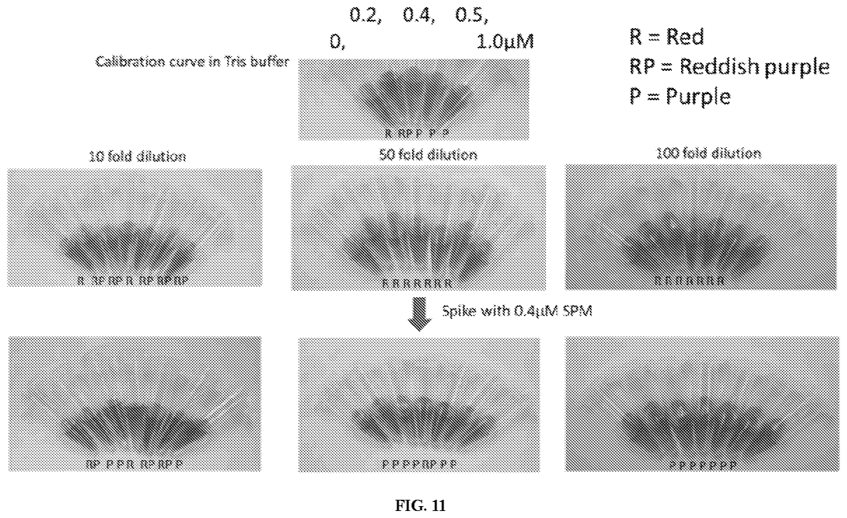

To optimize the detection conditions for Spm, a series of experiments were performed on sample clean up by dilution, which aimed to reduce the effect of interfering analytes present in the human urine sample. A colorimetric calibration of Spm in Tris buffer (5 mM Tris, 50 mM NaCl, 0.05% (w/w) NaN₃) with MUA-AuNPs was prepared from various Spm concentration (0, 0.2, 0.4, 0.5, 1.0 μM). Seven clinical urine samples were diluted by Tris buffer at different dilution factor (10-fold, 50-fold and 100-fold), followed by spiking of SPM for the visual color change (FIG. 11). For 10-fold dilution, most samples remain red even after the spiking of Spm, which indicated the detection of SPM was limited by the matrix effect of the sample. For 50-fold dilution, six out of seven samples were found purple after the spiking that showing improved Spm detection. For 100-fold dilution, all samples turned purple after the addition of SPM that demonstrated the 100-fold dilution showed the best efficiency of sample cleanup for more accurate SPM detection in urine.

To test the selectivity and specificity of the MUA-AuNPs described herein towards the target Spm, selectivity experiments are performed by testing the MUA-AuNPs across a range of polyamine and related molecules, followed by UV-Vis absorption measurement to study the aggregation profile of the MUA-AuNPs in the presence of different polyamines (all at 250 nM) (FIG. 9). The result shows that only the presence of Spm would lead to a red-shift of the absorbance and hence demonstrating the MUA-AuNPs is highly selective for the target Spm.

The following clinical urine samples were tested and served as examples for describing and illustrating the present invention. As such, they should not be construed to limit the scope of the invention.

### EXAMPLES

### Example 1. Detection of Urinary Spm in Cancerous Patient

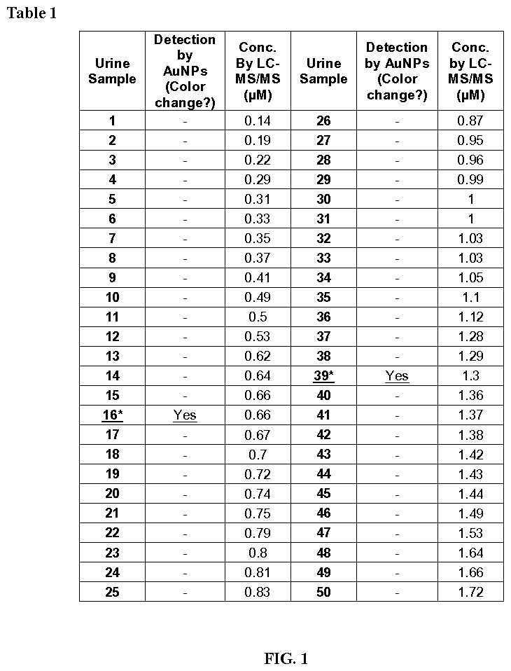

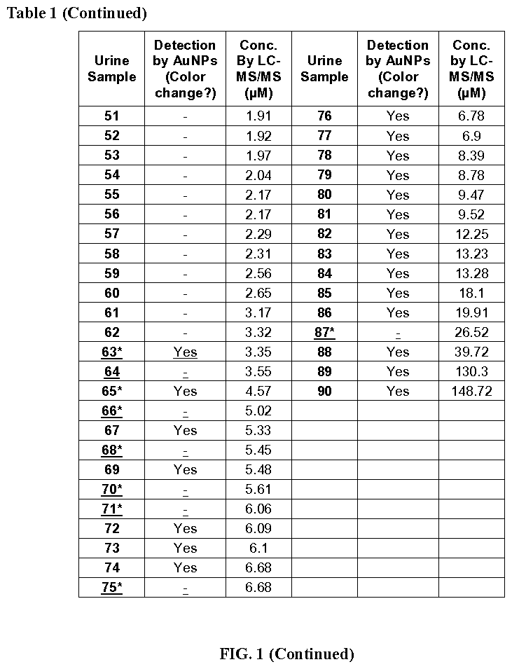

90 clinical urine samples obtained from cancer patients were analyzed by liquid chromatography-tandem mass spectrometry (LC-MS/MS) for the quantification of urinary Spm. These clinical urine samples (5 μL) were added to 470 μL of Tris-HCl buffer (5 mM, 50 mM NaCl, 0.05% (w/w) NaN₃) to prepare the samples. A solution of the MUA-AuNPs in water (calculated XX μM, 25 μL as prepared from HAuCl₄ (4.25 mg, 12.5 μmol) and trisodium citrate dihydrate (0.02 g, 68 μmol) and MUA (1 mg, 4.58 μmol) for the determination of Spm level in urine and urine matrix interference is determined to be equivalent to 250 nM Spm after 100-fold dilution by the buffer system. All analyses were done in 5 mM Tris-HCl buffer, 50 mM NaCl, 0.05% NaN₃, pH 7.2. The mixed samples are allowed to stand for 30 mins for saturated color change (if any). The cross-checking results are listed in the Table 1 (FIG. 1) with an accuracy of 80/90 by using a reported threshold point of 35 nM for cancer differentiation. Samples that did not give an expected color change are marked with asterisk (see Table 1).

What is claimed:

1. A method for detecting spermine (Spm) in a sample, the method comprising:
providing a sample;
contacting the sample with a plurality of gold nanoparticles (MUA-AuNPs) comprising citrate and 11-mercaptoundecanoic acid (MUA) thereby forming a test sample having a test color; and
detecting based on the test color the presence of Spm in the sample.

2. The method of claim 1, wherein the sample comprises urine obtained from a subject.

3. The method of claim 1, wherein the step of detecting based on the test color comprises visual inspection of the test color or determining the absorbance of the test sample using a spectrometer.

4. The method of claim 3 further comprising the step of comparing the test color with a color chart or color wheel prepared by using the interrelation between color of known concentrations of Spm in standard samples comprising MUA-AuNPs; or comparing the absorbance of the test sample obtained using a spectrometer with one or more calibration curves prepared using the interrelation between absorbance of known concentrations of Spm in standard samples comprising MUA-AuNPs; and determining the concentration of Spm in the sample.

5. The method of claim 2, wherein the method further comprises the step of comparing the test color to a color chart or color wheel prepared by using the interrelation between known concentrations of Spm in standard samples comprising MUA-AuNPs; or comparing the absorbance of the test sample using a spectrometer with one or more calibration curves prepared by using the interrelation between known concentrations of Spm in standard samples comprising MUA-AuNPs; determining the concentration of Spm in the sample; and based on the concentration of Spm in the sample determine if the subject has prostate cancer.

6. The method of claim 2 further comprising the step of:
providing a urine sample obtained from the subject; and
diluting the urine sample by 50 to 1,000 fold using an aqueous solvent thereby forming the sample.

7. The method of claim 1, wherein the aqueous solvent comprises tris(hydroxymethyl)aminomethane (Tris)-HCl and NaCl.

8. The method of claim 1, wherein the plurality of MUA-AuNPs are prepared according to a method comprising:
contacting HAuCl₄ with trisodium citrate thereby forming a plurality of citrate capped gold nanoparticles (citrate-AuNPs); and
contacting the plurality of citrate-AuNPs with 11-mercaptoundecanoic acid thereby forming the plurality of MUA-AuNPs.

9. The method of claim 8, wherein HAuCl₄ and trisodium citrate are contacted in a molar ratio between 1:3 to 1:15, respectively.

10. The method of claim 8, wherein the citrate-AuNPs and MUA are contacted in a molar ratio of 1:3 to 1:15, respectively.

11. The method of claim 2, wherein the after the step of contacting the sample with the plurality of MUA-AuNPs the test sample is incubated for at least 15 minutes before the step of detecting based on the test color the presence of Spm in the sample.

12. The method of claim 2, wherein after the step of contacting the sample with the plurality of MUA-AuNPs the test sample is incubated for 15 and 30 minutes before the step of detecting based on the test color the presence of Spm in the sample.

13. The method of claim 1, wherein the method comprises:
providing a sample comprising urine at a concentration of 0.1-2% (v/v), wherein the urine was obtained from a subject;
contacting the sample with a plurality of MUA-AuNPs comprising citrate and MUA thereby forming a test sample having a test color; and
detecting based on the test color the presence of Spm in the sample, wherein after the step of contacting the sample with the plurality of MUA-AuNPs, the test sample is incubated for 15 and 30 minutes before the step of detecting based on the test color the presence of Spm in the sample.

14. The method of claim 13, wherein the plurality of MUA-AuNPs have an average particle size of 1-20 nm.

15. The method of claim 13, wherein the sample comprises Tris-HCl and NaCl.

16. The method of claim 13, wherein the plurality of MUA-AuNPs are prepared according to a method comprising:
contacting HAuCl₄ with trisodium citrate in a molar ratio of 1:3 to 1:15, respectively thereby forming a plurality of citrate-AuNPs; and
contacting the plurality of citrate-AuNPs with MUA in a molar ratio of 1:3 to 1:15, respectively, thereby forming the plurality of MUA-AuNPs.

17. A method for diagnosing prostate cancer in a subject, the method comprising:
providing a sample comprising urine at a concentration of 0.1-2% (v/v), wherein the urine was obtained from the subject;
contacting the sample with a plurality of MUA-AuNPs comprising citrate and MUA thereby forming a test sample having a test color; and determining based on the test color whether the subject has prostate cancer, wherein after the step of contacting the sample with the plurality of MUA-AuNPs the test sample is incubated for 15 and 30 minutes before the step of determining based on the test color whether the subject has prostate cancer.

18. The method of claim 17, wherein the step of determining based on the test color whether the subject has prostate cancer comprises comparing the test color to a color chart or color wheel prepared by using the interrelation between known concentrations of Spm in standard samples comprising MUA-AuNPs; or comparing the absorbance of the test sample using a spectrometer with one or more calibration curves prepared by using the interrelation between known concentrations of Spm in standard samples comprising MUA-AuNPs; determining the concentration of Spm in the sample; and
based on the concentration of Spm in the sample determine if the subject has prostate cancer.

19. The method of claim 17, wherein the plurality of MUA-AuNPs are prepared according to a method comprising:
contacting HAuCl₄ with trisodium citrate in a molar ratio of 1:3 to 1:15, respectively thereby forming a plurality of citrate-AuNPs; and
contacting the plurality of citrate-AuNPs with MUA in a molar ratio of 1:3 to 1:15, respectively thereby forming the plurality of MUA-AuNPs.

20. The method of claim 17, wherein the MUA-AuNPs have an average particle size of 10-15 nm.
